(19) United States
(12) Patent Application Publication
(10) Pub. No.: US 2026/0026719 A1
(43) Pub. Date: Jan. 29, 2026

(54) SYSTEMS AND METHODS FOR CORRECTING AN OXYGEN SATURATION VALUE FOR PATHLENGTH ERRORS

(71) Applicant: Covidien LP, Mansfield, MA (US)

(72) Inventors: Scott James McGonigle, Loanhead (GB); Jacob D. Dove, Lafayette, CO (US); Pedro M. Elias Monteiro Gomes, Sobral de Monte Agraco (PT); Clark R. Baker, JR., Newman, CA (US); Dean Montgomery, Edinburgh (GB)

(21) Appl. No.: 19/243,527

(22) Filed: Jun. 19, 2025

Related U.S. Application Data
(60) Provisional application No. 63/675,785, filed on Jul. 26, 2024.

(51) Int. Cl. A61B 5/1455; A61B 5/00; A61B 5/1495; G16H 40/63

(52) U.S. Cl. CPC A61B 5/14552; A61B 5/1495; A61B 5/7225; A61B 5/742; G16H 40/63; A61B 2562/085

(57) ABSTRACT

A medical monitoring system including a sensor and a monitor. The sensor includes a sensor memory that stores a sensor identifier, a weight and bias, or any combination thereof. The monitor includes a port to communicatively couple to the sensor to receive a sensor signal, the sensor identifier, the weight and bias, or any combination thereof, a display, and monitor processing circuitry. The monitor processing circuitry to select a coefficient based on the sensor identifier, input the coefficient, the sensor signal, the weight and bias, or any combination thereof to a neural network to output a correction factor, and adjust a physiological parameter based on the correction factor.

### CROSS REFERENCE TO RELATED APPLICATION

The present application claims the benefit of U.S. Provisional Patent Application Ser. No. 63/675,785, filed on Jul. 26, 2024, the entire content of which is incorporated herein by reference.

### TECHNICAL FIELD

The present disclosure generally relates to medical monitoring devices (e.g., sensors) that adjust an oxygen saturation value based on an oxygen saturation correction to generate a corrected oxygen saturation value.

### BACKGROUND

Various medical monitoring devices may be used to monitor physiological characteristics of an individual. For example, various sensors may be used to measure temperature, pressure, oxygen, and other physiological characteristics of the individual. One such sensor, a pulse oximetry sensor, may be used to measure oxygen saturation levels in blood of the individual by utilizing wavelengths of light. In this manner, the pulse oximetry sensor may provide physiological parameters related to respiratory and circulatory systems of the individual.

In certain cases, physiological factors affecting light absorption and scattering, such as skin (e.g., tissue) pigmentation of a patient, skin thickness of the patient, skin abnormalities (e.g., scarring) of the patient, or sensor positioning on the patient may lead to errors in pulse oximetry readings. For example, skin pigmentation may contribute to errors in a blood oxygen saturation (SpO₂) value.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it may be understood that these statements are to be read in this light, and not as admissions of prior art.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, a medical monitoring system including a sensor and a monitor. The sensor includes a sensor memory that stores a sensor identifier, a weight and bias, or any combination thereof. The monitor includes a port to communicatively couple to the sensor to receive a sensor signal, the sensor identifier, the weight and bias, or any combination thereof, a display, and monitor processing circuitry. The monitor processing circuitry to select a coefficient based on the sensor identifier, input the coefficient, the sensor signal, the weight and bias, or any combination thereof to a neural network to output a correction factor, and adjust a physiological parameter based on the correction factor

In another embodiment, a method of operating a medical monitoring system, including receiving, at a processor and from a memory of a sensor, a sensor identifier, a weight and bias, a sensor signal, or any combination thereof, and selecting, via the processor, a coefficient based on the sensor identifier. The method also includes inputting, via the processor, the coefficient, the weight and bias, or any combination thereof to a neural network to output a correction factor, determining, via the processor, a physiological parameter based on the sensor signal, and adjusting, via the processor, the physiological parameter based on the correction factor.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a perspective view of an embodiment of a medical monitoring system configured to monitor oxygen saturation, in accordance with an aspect of the present disclosure;

FIG. 2 is a block diagram of an embodiment of the medical monitoring system of FIG. 1, in accordance with an aspect of the present disclosure;

FIG. 3 is a block diagram illustrating an example technique for generating a corrected oxygen saturation value that may be employed by the medical monitoring system of FIG. 1, in accordance with an aspect of the present disclosure;

FIG. 4 is a flow diagram of an embodiment of a method for adjusting an oxygen saturation value based on an oxygen saturation correction to generate the corrected oxygen saturation value, in accordance with an aspect of the present disclosure; and

FIG. 5 is a flow diagram of an embodiment of a method for generating a second oxygen saturation correction, in accordance with an aspect of the present disclosure.

### DETAILED DESCRIPTION OF THE INVENTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

It is presently recognized that it may be desirable to estimate and correct for pathlength errors due to pathlength variation of light emitted and detected by a sensor, such as pathlength variation of light that occurs due to various physiological factors of skin (e.g., skin pigmentation, skin thickness, skin abnormalities, skin damage) of a patient. Indeed, the physiological factors of the skin may result in different light absorption or scattering (e.g., within a spectral bandwidth of interest), which without embodiments disclosed herein, may result in error in an oxygen saturation (SpO₂) value determined and output by a monitor based on signals from the sensor. Certain existing systems may adjust the oxygen saturation value based on a predicted or estimated pigmentation of the skin of the patient. However, it is presently recognized that to improve accuracy, it may be desirable to adjust the oxygen saturation value based on one or more input signals (e.g., inputs) derived from the signals received from the sensor and a neural network to generate a corrected oxygen saturation value. Further, it may be desirable to display the corrected oxygen saturation value to enable an accurate reading of an oxygen saturation level of blood of the patient, which may enable appropriate health monitoring and management.

Accordingly, the present disclosure generally relates to systems and methods for adjusting an oxygen saturation value based on an oxygen saturation correction output by a neural network to generate a corrected oxygen saturation value. For example, a monitor of a medical monitoring system retrieves sensor information from a memory of a sensor of the medical monitoring system. The sensor information may include a sensor identifier (e.g., type of sensor, sensor family, sensor location) and/or one or more weights and biases. The monitor then performs coefficient selection to select one or more coefficients based on the sensor identifier. Additionally, the monitor inputs the one or more coefficients and/or the one or more weights and biases into the neural network. In some embodiments, the monitor may also receive one or more characterization parameters from the sensor. The one or more characterization parameters could include a total radiant flux from an LED of the sensor or a power transfer unit (PTU), which would account for the amount of light emitted from the LED and the efficiency of the photodiode to convert that light to a photocurrent. The one or more characterization parameters may be stored on the memory of the sensor and used as inputs into the neural network. For example, a PTU characterization value of the sensor (e.g., a manufactured sensor) would be measured and recorded in the memory of the sensor. Therefore, when plugged into the monitor (e.g., an oximeter) the PTU characterization value may be read by the monitor and input into the neural network.

In addition, the monitor receives and/or determines one or more input signals (e.g., ratio of alternating current (AC) components, ratio of direct current (DC) components, a filtered ratio-of-ratios, red percent modulation, infrared (IR) percent modulation, and any other suitable input). The monitor may input (e.g., provide) the one or more input signals to the neural network. The neural network may perform analysis and output an oxygen saturation correction based on the one or more coefficients, the weight and bias, the one or more input signals, and/or the PTU characterization. Further, the monitor adjusts an oxygen saturation value (e.g., calculated based on signals received from the sensor) based on the oxygen saturation correction output by the neural network to generate a corrected oxygen saturation value. In this manner, the oxygen saturation correction may account for pathlength errors due to light scattering and absorption based on the physiological factors.

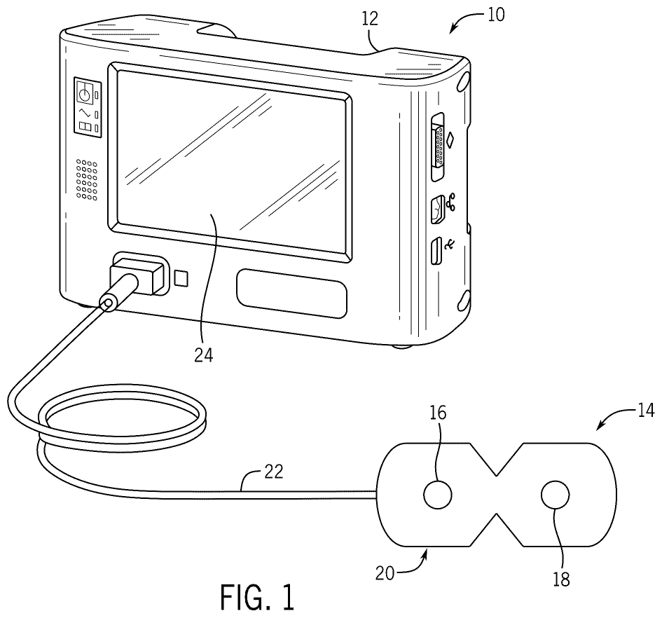

With the foregoing in mind, FIG. 1 is a perspective view of an embodiment of a medical monitoring system 10 that includes a patient monitor 12 (also referred to herein as “the monitor 12”) that may be used in conjunction with a medical sensor 14 (also referred to herein as “the sensor 14”). In the illustrated example, the monitor 12 is a pulse oximetry monitor and the sensor 14 is a pulse oximetry sensor. In such cases, the monitor 12 is configured to process photoplethysmography (PPG) signals to calculate oxygen saturation (SpO₂). It should be appreciated that the medical monitoring system 10 may be configured to obtain any of a variety of medical measurements and the techniques described herein may be adapted for use with any variety of monitors and sensors. By way of non-limiting example, in some embodiments, the monitor 12 may include a regional oximeter and the sensor 14 may include a regional saturation sensor. In such cases, the monitor 12 is configured to process the PPG signals to calculate regional oxygen saturation (rSO2). As other non-limiting examples, the sensor 14 may be a thermometer to measure body temperature, an electrocardiography sensor to measure heart rate and other heart parameters, an electroencephalography sensor to measure brain activity, a glucose sensor to monitor glucose, a blood pressure sensor to measure blood pressure, or any other suitable type of sensor, and the monitor 12 may be configured to process the signals received from the sensor 14. Additionally, although the depicted embodiments illustrate the sensor 14 configured for use on a patient's finger, it should be understood that the sensor 14 may be adapted for use at other tissue locations, such as a forehead, temple, earlobe, toe, foot, heel, ankle, stomach, chest, back, neck, wrist, thigh, or any other suitable measurement site.

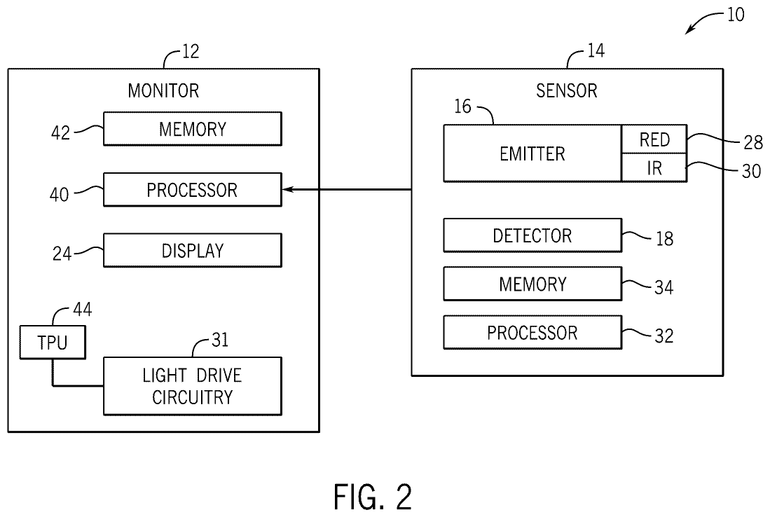

In FIG. 1, the sensor 14 includes one or more emitters 16 (collectively referred to herein as “the emitter 16” for convenience) and one or more detectors 18 (collectively referred to herein as “the detector 18” for convenience). The emitter 16 emits wavelengths of light that passes through blood perfused tissue, and the detector 18 detects the light as reflected or transmitted by the tissue. Additional details regarding the emitter 16 and the detector 18 will be described below with respect to FIG. 2. In certain embodiments, the sensor 14 may include sensing components in addition to, or instead of, the emitter 16 and the detector 18.

The sensor 14 includes a sensor body 20 that may include multiple layers, such as a backing, adhesives, and so on. The sensor body 20 may also include or support a flexible circuit with various components. In certain embodiments, the medical monitoring system 10 may include multiple sensors 14 at multiple locations.

The sensor 14 is communicatively coupled to the monitor 12. In the illustrated embodiment, the sensor 14 is coupled to the monitor 12 via a cable 22. The cable 22 may interface directly with the sensor 14 and may include multiple conductors (e.g., wires) to transmit signals and/or receive signals. Additionally or alternatively, the sensor 14 may communicate with the monitor 12 wirelessly (e.g., the sensor 14 and the monitor 12 include wireless transceivers configured to communicate via any suitable wireless protocol). For example, the sensor 14 may include a transceiver that enables wireless signals to be transmitted to and/or received from an external device (e.g., the monitor 12). Additionally, the multiple conductors or the transceiver may transmit a raw digitized detector signal, a processed digitized detector signal, or a calculated physiological parameter, as well as any data (e.g., sensor identifier data, neural network data, coefficient data, power transfer unit (PTU) characterization data) that may be stored in the sensor 14.

In operation, the monitor 12 may receive a signal from the sensor 14, and the monitor 12 may be configured to calculate or measure one or more physiological parameters based on the signal. In particular, the monitor 12 may include a processor configured to execute code (e.g., stored in a memory of the monitor 12 or received from another device) for filtering and processing the signal from the sensor 14 to calculate physiological parameters, such as oxygen saturation. The monitor 12 may additionally or alternatively calculate any variety of physiological parameters, such as arterial blood oxygen saturation, regional or tissue oxygen saturation, pulse rate, respiration rate, blood pressure, blood pressure characteristic measure, autoregulation status, brain activity, temperature, or any other suitable physiological parameter.

Additionally, as illustrated in FIG. 1, the monitor 12 includes a display 24 configured to display one or more calculated physiological parameters, such as the oxygen saturation and/or a corrected oxygen saturation (e.g., corrected based on one or more input signals and one or more coefficients input into a neural network to output a correction value, as described in more detail herein). The display 24 may also display other information, such as instructions to charge the sensor 14, alarm indications, settings, and so forth. In certain embodiments, the display 24 may be a touch screen display. The monitor 12 may include various input components, such as the touch screen display, knobs, switches, keys and keypads, buttons, and so forth, to provide for operation and configuration of the monitor 12. The monitor 12 may also include one or more indicator lights and one or more speakers. The monitor 12 may also include additional slot(s) or wireless interfaces (e.g., channels) to connect to additional devices, such as additional sensors to monitor additional physiological parameters of the patient and/or to monitor physiological parameters of other patients at one time.

Furthermore, one or more functions of the monitor 12 disclosed herein may also be implemented directly in the sensor 14, or by any other suitable device. For example, in some embodiments, the sensor 14 may include one or more processing components configured to calculate physiological parameters, such as oxygen saturation. Additionally or alternatively, in some embodiments, the sensor 14 may include the one or more processing components configured to calculate the correction value. The sensor 14 may have varying levels of processing power, and may output data in various stages to the monitor 12. For example, in some embodiments, the data output to the monitor 12 may be analog signals, such as detected light signals (e.g., pulse oximetry signals or regional saturation signals), or processed data.

Further, in some embodiments, the sensor 14 may include a battery to provide power to components of the sensor 14. For example, the sensor 14 may be configured to operate in a wireless mode and, at times, may not receive power from the monitor 12 while operating in the wireless mode. In some embodiments, the battery may be a rechargeable battery such as, for example, a lithium ion, a lithium polymer, a nickel-metal hydride, a nickel-cadmium battery, or any other suitable rechargeable battery. In other embodiments, any suitable power source may be utilized, such as, one or more capacitors or an energy harvesting power supply (e.g., a motion generated energy harvesting device, thermoelectric generated energy harvesting device, or any other suitable energy harvesting power supply).

Turning to FIG. 2, a simplified block diagram of the medical monitoring system 10 is illustrated in accordance with an embodiment. As described herein, the sensor 14 includes the emitter 16 and the detector 18. The emitter 16 includes two light emitting diodes (LEDs) that are configured to emit at least two wavelengths of light, e.g., a red LED 28 configured to emit wavelengths of light within the red spectrum and an infrared (IR) LED 30 configured to emit wavelengths of light within the infrared or near infrared spectrum. In one embodiment, the LEDs 28, 30 emit light in a range of about 600 nanometers (nm) to about 1000 nm. In one embodiment, the red LED 28 is configured to emit light between approximately 600 nm and 735 nm, and the IR LED 30 is configured to emit light between approximately 800 nm and 1000 nm. It should be noted that the emitter 16 may also transmit 3, 4, or 5 or more wavelengths of light in any suitable application.

As discussed in more detail herein, a light drive circuitry 31 of the monitor 12 may provide respective drive currents to the LEDs 28, 30 to cause the LEDs 28, 30 to emit respective wavelengths of light. The emitter 16 emits light that passes through blood perfused tissue, and the detector 18 detects the light as reflected or transmitted by the tissue. The emitter 16 and the detector 18 may be arranged in a transmission configuration or a reflectance configuration with respect to one another. In the transmission configuration, the light enters the detector 18 after passing through the tissue of the patient. In the reflectance configuration, the light is reflected by elements in the tissue of the patient to enter the detector 18. In any case, the detector 18 may generate a signal (e.g., PPG signal) indicative of an intensity of the light received at the detector 18, and the detector 18 may send the signal to the monitor 12.

A signal representing light intensity versus time or a mathematical manipulation of this signal (e.g., a scaled version thereof, a log taken thereof, a scaled version of a log taken thereof) may be referred to as the PPG signal. Additionally, the term “PPG signal,” as used herein, may also refer to an absorption signal (e.g., representing an amount of light absorbed by the tissue) or any suitable mathematical manipulation thereof. The amount of light detected or absorbed may then be used to calculate any of a number of physiological parameters, including oxygen saturation (e.g., the saturation of oxygen in pulsatile blood, SpO₂), an amount of a blood constituent (e.g., oxyhemoglobin), and/or a physiological rate (e.g., pulse rate or respiration rate; when each individual pulse or breath occurs).

For oxygen saturation, red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, from curve fitting, or other interpolative techniques.

As shown, the sensor 14 also includes one or more processors 32 (collectively referred to herein as “the processor 32” for convenience) and one or more memory devices 34 (collectively referred to herein as “the memory 34” for convenience). The memory 34 may include or be an add-only memory, a rewriteable integrated circuit, or other suitable memory type. The processor 32 may be part of a processing system (e.g., processing circuitry) to perform operations and execute instructions stored in the memory 34. Further, at least a portion of the memory 34 is erasable and reprogrammable. For example, the memory 34 may include an erasable programmable read-only memory (EPROM), which is erasable and reprogrammable, and thus enables a user to provide inputs to write data to it.

The memory 34 stores sensor information about the sensor 14, such as a type of sensor, a sensor identifier, a weight and bias, one or more characterization parameters, such as a power transfer unit (PTU) characterization or a total radiant flux from the LEDs 28, 30, and/or calibration information. For example, the sensor identifier, the weight and bias, and/or the PTU characterization may be stored on the EPROM to enable modification to the sensor information (e.g., without updating firmware of the sensor 14). In this manner, updated sensor information may be written to the EPROM and enable overwrite of previously stored sensor information on the EPROM. In some embodiments, the sensor information may be stored on an EPROM separate from the sensor 14 and coupled to the sensor 14 via a cable or dongle.

In certain embodiments, the sensor 14 may communicate or provide the sensor information to the monitor 12. Together, the sensor information, such as the sensor identifier, may enable the monitor 12 to select one or more coefficients to input into a neural network to generate an oxygen saturation correction value.

The sensor identifier may include information (e.g., data) associated with a type of sensor, a sensor location (e.g., on the patient), and/or a sensor family. For example, the sensor type or family may include a group of sensors that share a particular set of characteristics, such as a same or similar type of emitter 16 or detector 18, or a same or similar location of placement on the body (e.g., forehead, finger, foot), or a same or similar patient population (e.g., adults, pediatrics, neonatal). In certain embodiments, the sensor identifier may be provided to the monitor 12 to enable the monitor to select the one or more coefficients to input into the neural network based on the sensor identifier.

The weight may include one or more weights, wherein each weight includes a numerical value (e.g., −3, −1.25, −1, 0, 1, 2, 2.25, 3, 4, 5, or more) associated with connections between neurons of the neural network. Further, the weight may determine strength of the connections and an amount of influence an input from each neuron in a previous layer has on a final output of an output layer. The bias may include one or more additional parameters, such as a constant value, added to the output layer neurons to enable the neural network to account for an imbalance or offset in the input data. In certain embodiments, the weight and bias may be provided to the monitor 12 to enable the monitor 12 to input the weight and bias into the neural network.

In addition, the PTU characterization may involve analysis of various parameters associated with the PTU of the sensor 14 to determine performance and behavior of the PTU. For example, the PTU may include a normalized measurement of power response of the sensor 14 based on optical power of the emitter 16, bandaging, and responsivity of the detector 18. The PTU characterization may be provided to the monitor 12 to enable the monitor to input the PTU characterization into the neural network.

It should be appreciated that, in certain embodiments, the memory 34, such as the EPROM, may store at least a portion of data associated with a neural network and communicate such data to the monitor 12. For example, the memory 34, such as the EPROM, may store the sensor identifier that is used to select the one or more coefficients for the neural network and/or may store the weight and bias for the neural network. By storing the sensor identifier and/or the weight and bias on the sensor 14, the sensor 14 may be considered to store at least part of the neural network on the sensor 14. Thus, updates to the sensor identifier and/or the weight and bias on the sensor 14 may effectively result in updates to the neural network (e.g., via connection of the sensor 14 to the monitor 12; without separately updating or replacing the monitor 12).

In this manner, the portion of data associated with the neural network may be updated (e.g., removed, replaced, rewritten, adjusted) in the memory 34 of the sensor 14 at a later time (e.g., in the future) to effectively update the neural network. For example, the portion of data associated with the neural network (e.g., the sensor identifier and/or the weight and bias) may be removed (e.g., erased) and/or replaced (e.g., replaced with updated data, such as an updated sensor identifier and/or updated weight and bias) via the EPROM of the sensor 14 to update the sensor identifier and/or the weight and bias, and to effectively update the neural network. In certain embodiments, the portion of data associated with the neural network may be updated in the memory 34 of the sensor 14 based on introduction of a new sensor family (e.g., sensor type) at the later time, a change in an existing sensor family (e.g., new emitters) at the later time, and/or recognition of improvements to the neural network at the later time (e.g., data collected and analyzed may indicate that updated coefficients, weight, and/or bias would provide more accurate results via the neural network).

When accessed by the monitor 12, the sensor information about the sensor 14 may enable the monitor 12 to process the signal (e.g., based on the light detected at the detector 18 of the sensor 14) received from the sensor 14 to calculate oxygen saturation and/or other physiological parameters. As shown, the monitor 12 includes one or more processors 40 (collectively referred to herein as “the processor 40” for convenience), one or more memory devices 42 (collectively referred to herein as “the memory 42” for convenience), and the display 24. The processor 40 may be part of a processing system (e.g., processing circuitry) to perform operations and execute instructions stored in the memory 42. As used herein, the monitor 12 may refer to and/or include a circuit board (e.g., a printed circuit board (PCB), electronic board), as well as additional computing components. For example, the circuit board may process the sensor information and the signal from the sensor 14 (e.g., to implement processing techniques disclosed herein), while the additional computing components may include multi-purpose computing components to combine data from multiple sources and/or to display information for visualization by a user. Thus, the processor 40 may refer to and/or include one or more processors of the circuit board, as well as one or more processors of the additional computing components. Further, the circuit board and the additional computing components may be included in one housing (e.g., a monitor housing) or may be included in separate housings.

The processor 40 may process the signal received from the sensor 14, such as by performing synchronized demodulation, amplification, and filtering of the signal. The processor 40 may process the signal received from the sensor 14 to calculate one or more physiological parameters, such as the oxygen saturation, using various algorithms. Physiological parameter coefficients utilized in the algorithms may be accessed by the processor 40 from the memory 34 or determined by the processor 40 based at on the sensor information of the sensor 14 (e.g., the calibration information), for example. In some embodiments, the monitor 12 may already have the physiological parameter coefficients stored in the memory 42 of the monitor 12

As shown, the monitor 12 includes a time processing unit (TPU) 44, which may be controlled by the processor 40 and is configured to provide timing control signals to the light drive circuitry 31 and optionally to other parts of the medical monitoring system 10. The light drive circuitry 31 may control when the red LED 28 and the IR LED 30 are illuminated and/or a drive current provided to the red LED 28 and the IR LED 30. It should be appreciated that one or more functions or components of the monitor 12 disclosed herein may also be implemented directly in the sensor 14, or by any other suitable device.

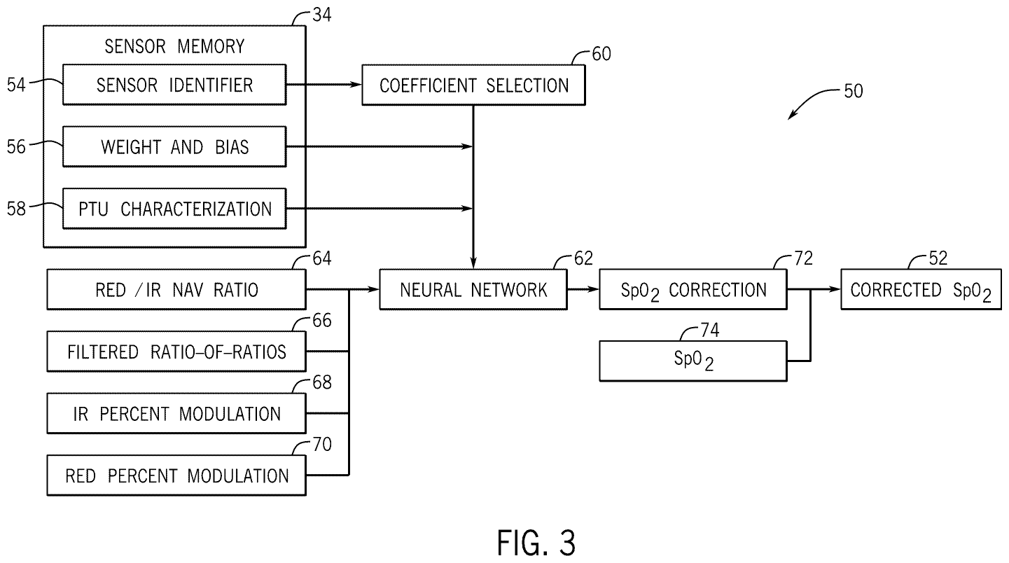

FIG. 3 is a block diagram illustrating an example technique 50 (e.g., algorithm) for generating a corrected oxygen saturation value 52 (e.g., a corrected SpO₂), in accordance with an aspect of the present disclosure. While the technique 50 is described herein as being performed or utilized by the monitor 12 of the medical monitoring system of FIG. 1, it should be noted that the technique 50 may be performed or utilized by any other suitable device, such as the sensor 14 employed in the medical monitoring system 10 of FIG. 1 or any suitable computing system (e.g., cloud computing system).

In operation, the monitor 12 receives sensor information from the memory 34 of the sensor 14. For example, the sensor information may include a sensor identifier 54 and/or a weight and bias 56 stored in the memory 34 (e.g., an EPROM memory) of the sensor 14. The sensor identifier may include information (e.g., data) associated with a type of sensor, a sensor location, and/or a sensor family. The type of sensor or sensor family may include any suitable group of sensors that share a particular set of characteristics. For example, the type of sensor or sensor family may include a transmittance sensor, a reflectance sensor, a finger clip sensor, an earlobe sensor, a disposable sensor, a multi-site sensor, or any other suitable type of sensor. Additionally or alternatively, the sensor type or sensor family may include a type of emitter 16 (e.g., one or more wavelengths emitted by the emitter 16) and a type of detector 18. The sensor location may include a forehead, a temple, an earlobe, a foot, a wrist, a hand, a finger, or any other suitable location for the measurement site of the patient.

The weight may include one or more weights, and each weight may include a numerical value, such as −4, −2.5, −1, 0, 1, 2, 3, 3.5, 4, 5, or more, associated with connections between neurons of a neural network 62 (e.g., machine learning (ML) model, artificial intelligence (AI) model). For example, the weight may be associated with a strength of each of the connections and may modify (e.g. influence) a neuron's output on a neuron's input. Indeed, the weight may depict a significance (e.g., importance) of each portion of data (e.g., a feature) provided to the neural network 62. The bias (e.g., offset, threshold) may include one or more biases, and each bias may include a constant value (e.g., a positive number or a negative number) associated with or added to each portion of data provided to the neural network 62. For example, the bias may enable an activation function of each portion of data to shift, which may modify the output of each neuron.

In some embodiments, the monitor 12 also receives a PTU characterization 58. As an example, the PTU characterization 58 may involve placement of the sensor 14 on a characterized optical phantom or integrating sphere to obtain a measure of optical efficiency of the emitter 16 (or multiple emitters 16) and a power ratio of the sensor 14. Therefore, the PTU characterization 58 may include a normalized measurement of a power response of the sensor 14 based on optical power of the emitter 16, bandaging of the sensor 14, and responsivity of the detector 18. In certain embodiments, the monitor 12 inputs the PTU characterization 58 into the neural network 62.

In certain embodiments, the monitor 12 performs coefficient selection 60 (e.g., selects or determines one or more coefficients) based on the sensor identifier 54. The one or more coefficients may be employed by the neural network 62 to adjust strength of connections between the neurons. In certain embodiments, multiple coefficients (e.g., multiple sets of coefficients, such as −4.5, −2, −1, 0, 2, 2.5, 3, 4, 4.5, 5, 6, or more sets of coefficients) may be stored in the memory 42 of the monitor 12. Therefore, the monitor 12 may perform the coefficient selection 60 by selecting from the multiple coefficients stored in the memory 42 based on the sensor identifier 54. In certain embodiments, the monitor 12 may employ one or more algorithms to calculate or determine the one or more coefficients based on the sensor identifier 54. In any case, the monitor 12 provides the one or more coefficients selected at the coefficient selection 60 to the neural network 62.

Advantageously, as data is collected over time, the sensor information may be rewritten and updated in the memory 34 (e.g., the EPROM memory) of the sensor 14 based on the data collection. For example, the data that is collected over time may indicate that it would be desirable to make certain adjustments to the one or more coefficients to affect outputs of the neural network 62. In such cases, the sensor information may be rewritten and updated in the memory (e.g., the EPROM memory) of the sensor 14 to result in the certain adjustments to the one or more coefficients to affect outputs of the neural network 62. Similarly, as changes are made to the sensor 14 (e.g., new type of the sensor 14, new emitter incorporated to the sensor 14, and so forth), the sensor information may be rewritten and updated in the memory 34 (e.g., the EPROM memory) of the sensor 14 to prompt selection of the one or more coefficients that are appropriate for the sensor 14. It should be appreciated that, in certain embodiments, the one or more coefficients may be stored and/or updated in the memory 34 of the sensor (e.g., the EPROM memory). In such cases, the sensor 14 may provide the one or more coefficients to the monitor 12 for the coefficient selection 60.

In addition, the monitor 12 receives and/or derives one or more input signals (e.g., based on the signals received from the sensor 14 and based on the light detected at the detector 18 of the sensor 14). The one or more input signals may include a red/infrared (IR) nanoamperes virtual (nAv, virtual here refers to a normalization to account for varying LED drive currents) ratio 64 (e.g., a ratio of direct current (DC) components), a filtered ratio-of-ratios 66, an IR percent modulation 68 (e.g., IR perfusion index), and/or a red percent modulation 70 (e.g., red perfusion index). The red/IR nAv ratio 64 may include a ratio of Red_{DC} nAv to IR_{DC} nAv (e.g., (Redpc nAv)/(IR_{DC} nAv)), and may be associated with a photocurrent received by the detector 18 of the sensor 14 from the emitter 16 (e.g., after canceling ambient light). Therefore, a photocurrent may be associated with a metric of light absorption at a measurement site (e.g., location) of the sensor 14. The Redpc may refer to a constant part of light absorption at a red wavelength of the sensor 14. Moreover, the IR_{DC} may refer to a constant part of the light absorption at an IR wavelength and may be associated with a gain normalized IR signal intensity.

The filtered ratio-of-ratios 66 may include a ratio of Red_{AC} to Redpc (e.g., Red_{AC}/Redpc) divided by a ratio of IR_{AC} to IR_{DC} (e.g., IR_{AC}/IR_{DC}). The Red_{AC} may refer to a varying part of the light absorption at the red wavelength, which may be associated with a pulsatile flow of arterial blood. The IR_{AC} may refer to a varying part of the light absorption at the IR wavelength, which may be associated with the pulsatile flow of arterial blood. The IR percent modulation 68 may be associated with a percentage of modulation in intensity of the IR light emitted by the emitter 16 of the sensor 14, which may be detected by the detector 18 after passing through the tissue. The red percent modulation 70 may be associated with a percentage of modulation in intensity of the red light emitted by the emitter 16 of the sensor 14, which may be detected by the detector 18 after passing through the tissue. The IR percent modulation 68 and the red percent modulation 70 may indicate an amount of blood in the tissue of the patient at the site of the sensor 14.

The monitor 12 inputs (e.g., provides, feeds) the red/IR nAv ratio 64, the filtered ratio-of-ratios 66, the IR percent modulation 68, and/or the red percent modulation 70 into the neural network 62. It should be noted that any other suitable signal may be included or substituted as the inputs to the neural network 62. For example, the inputs may also include the calculated oxygen saturation value, a pulse rate (e.g., beats per minute), skewness, pulse amplitude, a pulse rate, and/or other input(s).

Additionally or alternatively, the inputs may include an additional one or more wavelengths of light and/or light detection at a different location (e.g., via the detector 18 or a separate detector included in the sensor 14). For example, at least one additional LED, such as a third LED, may be added to the sensor 14 to provide additional data (e.g., information) on light scattering and absorption. Indeed, the sensor 14 may employ two wavelengths of a larger length (e.g., 850 nm and 900 nm; emitted by the IR LED 30 and the third LED, respectively), which may be affected by the light scattering. Additionally or alternatively, the sensor 14 may include at least one additional detector, such as a second detector, to collect one or more optical signals that may have traveled along one or more different pathlengths (e.g., as compared to pathlengths to the detector 18). As such, the one or more input signals may include three nAv ratios 64. For example, a first nAv ratio 64 may include a ratio of the red LED 28 to the IR LED 30 (e.g., LED₁/LED₂), a second nAv ratio 64 may include a ratio of the IR LED 30 to the third LED (e.g., LED₂/LED₃), and a third nAv ratio 64 may include a ratio of the third LED to the red LED 28 (LED₃/LED₁).

By including the second detector, a ratio at each of and/or between the first detector 18 and the second detector may be calculated. For example, the ratios may include a ratio of the red wavelength to the IR wavelength at each of the first detector 18 and the second detector (e.g., (Red/IR)_{Detector 1}, (Red/IR)_{Detector 2}), a ratio of the red wavelength at the first detector 18 to the red wavelength at the second detector (e.g., (Red_{Detector 1}/Red_{Detector 2})), a ratio of the IR wavelength at the first detector 18 to the IR wavelength at the second detector (e.g., (IR_{Detector 1}/IR_{Detector 2})), and a ratio of the red wavelength to the IR wavelength at the first detector 18 to the red wavelength to the IR wavelength at the second detector (e.g., (Red/IR)_{Detector 1}/(Red/IR)_{Detector 2})).

In some embodiments, the monitor 12 may apply normalization terms to at least one or all of the one or more input signals. For example, the monitor 12 may determine a square root of each of the inputs to normalize each of the inputs. As another example, the monitor 12 may apply a logarithmic conversion to each of the inputs to normalize each of the inputs. Additionally or alternatively, the monitor 12 may perform filtering or conversions on the inputs prior to providing the inputs to the neural network 62. The monitor 12 may filter or convert the inputs by pre-processing (e.g., normalizing, scaling, encoding, and so on) input data associated with the inputs before providing the inputs to the neural network 62. By filtering or converting the input data associated with the inputs, the monitor 12 may provide stability to an oxygen saturation correction 72 output by the neural network 62.

As illustrated in FIG. 3, the one or more inputs, including the red/IR nAv ratio 64, the filtered ratio-of-ratios 66, the IR percent modulation 68, and/or the red percent modulation 70, are input into the neural network 62 to for processing the one or more inputs through a number of layers of the neural network 62. As an example, the neural network 62 may include one or more neurons (e.g., nodes) in a layer of the neural network 62, which may receive the one or more inputs, apply the weight and bias 56 and/or the one or more coefficients to the inputs, and produce one or more outputs. In some embodiments, the one or more outputs may be used in an additional one or more layers of the neural network 62. The process performed by the neural network 62 may continue until a final output layer of the neural network 62 is reached. The output layer of the neural network 62 may then output (e.g., produce) the oxygen saturation correction 72 (e.g., SpO₂ correction).

The oxygen saturation correction 72 (e.g., correction factor) may be expressed as a point value (e.g., a percentage value). Further, the point value may include a threshold (e.g., a cap) for a maximum point value and/or a threshold for a minimum point value. For example, the threshold for the maximum point value may be equal to plus or minus four points (e.g., +/−4 points) and the threshold for the minimum point value may be equal to plus or minus half a point (e.g., +/−0.5 point). Thus, the oxygen saturation correction 72 will be applied to adjust an oxygen saturation value 74 to generate the corrected oxygen saturation value 52 as long as the oxygen saturation correction 72 is less than or equal to the threshold for the maximum point value and greater than or equal to the threshold for the minimum point value (e.g., an increase between 0.5 to 4 points (inclusive) or a decrease between −0.5 to −4 points (inclusive); the oxygen saturation correction 72 will result in the corrected oxygen saturation value 52 within +/−0.5 to +/−4 points of the oxygen saturation value 74). It should be noted that any suitable number may be used as the threshold for the maximum value and the threshold for the minimum value. Indeed, the threshold for the maximum point value and the threshold for the minimum point value may include +/−0, 0.5, 1, 2, 3, 4, 5, or more. The threshold for the maximum point value may block adjustment to the oxygen saturation value 74 based on unexpectedly large oxygen saturation correction 72, as the unexpectedly large oxygen saturation correction 72 may be due to processing errors with the neural network 62 or some other factor separate from the patient (e.g., error with the sensor 14). Similarly, the threshold for the minimum point value may block adjustment to the oxygen saturation value 74 based on a small oxygen saturation correction 72, as the small oxygen saturation correction 72 may be not be clinically significant, for example. In certain embodiments, the threshold for the maximum point value and/or the threshold for the minimum point value may be set by a clinician (e.g., via input to the monitor 12), may be set by the monitor 12 based on patient data (e.g., patient disease state input to the monitor 12 and/or access from patient records; the oxygen saturation values 74 over time during a patient monitoring session and/or prior patient monitoring sessions; other physiological parameters of the patient), may be stored in the memory 42 of the monitor 12, and/or may be stored in the memory 34 of the sensor (e.g., the EPROM memory) of the sensor 14. Additionally, in some embodiments, the oxygen saturation correction 72 may include a continuous oxygen saturation correction 72 (e.g., performed continuously or repeatedly over time, such as each time the monitor 12 calculates the oxygen saturation value 74 based on the signals from the sensor 14). As shown in FIG. 3, the oxygen saturation correction 72 output by the neural network 62 is a correction for the calculated oxygen saturation value 74. It should be appreciated that the oxygen saturation correction 72 may be a correction for the ratio-of-ratios 66 (e.g., shift a calibration curve for conversion of ratio-of-ratios to oxygen saturation values, such as to generate the corrected oxygen saturation value 52 based on the signals received from the sensor 14).

As described herein, the monitor 12 is configured to process PPG signals from the sensor 14 to calculate the oxygen saturation value 74 (e.g., SpO₂). The monitor 12 then adjusts (e.g., updates) the oxygen saturation value 74 based on the oxygen saturation correction 72 to generate the corrected oxygen saturation value 52. For example, the monitor 12 may add the oxygen saturation correction 72 to the oxygen saturation value 74. In this manner, the monitor 12 may employ data (e.g., the one or more input signals) obtained at the measurement site of the sensor 14 to adjust the oxygen saturation value 74 and generate the corrected oxygen saturation value 52. In certain embodiments, the monitor 12 may average, filter, or smooth the oxygen saturation correction 72 over a period of time to reduce noise, and thus, inaccurate predictions based on the noise.

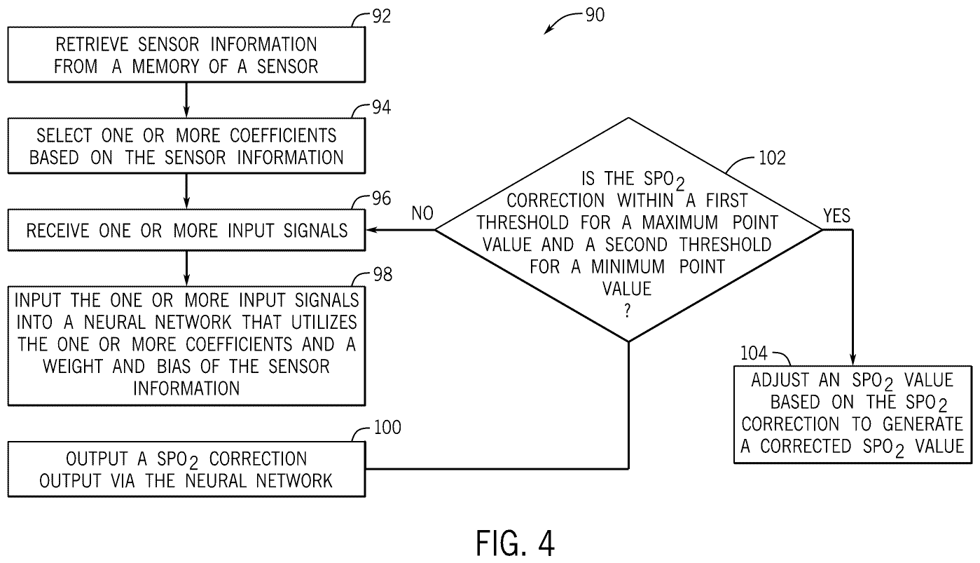

The monitor 12 may display (e.g., present) the corrected oxygen saturation value 52. For example, the monitor 12 may render a numerical value representative of the corrected oxygen saturation value 52 on the display 24 of the monitor 12. It should be noted that the example technique 50 described herein is merely illustrative, and any suitable number of inputs and type of inputs may be input into the neural network 62 to generate the oxygen saturation correction 72. Additional details regarding adjustment of the oxygen saturation value 74 to generate the corrected oxygen saturation value 52 will be described below with respect to FIG. 4.

FIG. 4 is a flow diagram of a method 90 for adjusting an oxygen saturation value based on an oxygen saturation correction to generate a corrected oxygen saturation value, in accordance with an aspect of the present disclosure. The method 90 disclosed herein includes various steps represented by blocks. It should be noted that at least some steps of the method 90 may be performed as an automated procedure by a system, such as the medical monitoring system 10 of FIG. 1 or a cloud computing system. Further, certain steps or portions of the method 90 may be performed by certain devices, such as the monitor 12 (e.g., the processing system that includes the processor 40) and/or the sensor 14 (e.g., the processing system that includes the processor 32). It should be appreciated that “the processing system” as used herein may refer to components (e.g., the processor 40, the processor 32) in the monitor 12, the sensor 14, or both. Although the flow diagram illustrates the steps in a certain sequence, it should be understood that the steps may be performed in any suitable order and certain steps may be carried out simultaneously, where appropriate.

At block 92, the processor of the monitor retrieves sensor information from a memory (e.g., EPROM memory) of a sensor. The sensor information may include a sensor identifier and/or a weight and bias. The sensor identifier may include data associated with a type of sensor, a sensor location, and/or a sensor family. Moreover, the weight may include one or more weights, and each weight may include a numerical value and may be associated with one or more connections between neurons of a neural network. The bias may include one or more biases, and each bias may include a constant value, which may be added to any suitable portion of data provided to the neural network. As such, the weight and bias may enable an output (e.g., product) of the neural network to include the weight to combine one or more inputs input into the neural network based on one or more features embedded in training of the neural network. Moreover, in some embodiments, the sensor information may also include a PTU characterization, which may be associated with a normalized measurement of a power response of a sensor based on optical power of an emitter of the sensor.

At block 94, the processor of the monitor selects (e.g., selects or determines) one or more coefficients based on the sensor information. In particular, the processor of the monitor selects the one or more coefficients based on the sensor identifier included in the sensor information. Further, at block 96, the processor of the monitor receives and/or derives one or more input signals. For example, the one or more input signals may include a ratio of DC components (e.g., ratio of received red light to received IR light), a ratio of AC components (e.g., ratio of red light modulation to IR light modulation), ratio-of-ratios (e.g., filtered ratio-of-ratios, perfusion index), a calculated oxygen saturation value (e.g., calculated based on a PPG signal from the sensor), and/or a calibration measurement. For example, the calibration measurement may be based on one or more optical properties of one or more emitters and/or a detector, such as a PTU ratio.

At block 98, the processor of the monitor inputs the one or more input signals into a neural network that utilizes the one or more coefficients and the weight and bias included in the sensor information. The coefficients may influence or control strength of one or more connections between neurons of the neural network. For example, the one or more coefficients may influence or control influence of each of the one or more input signals on a final output of the neural network. The weight may modify a neuron's output on a neuron's input. Indeed, the weight may depict a significance of each portion of data provided to the neural network. The bias may include a constant value associated with or added to each portion of data provided to the neural network. The final output of the neural network may include an oxygen saturation correction. The processor of the monitor may apply the oxygen saturation correction to the calculated oxygen saturation value to correct inaccuracies or errors of the calculated oxygen saturation value (e.g., inaccuracies or errors due to parameters of skin of the patient).

Indeed, at block 100, the processor of the monitor outputs the oxygen saturation correction output via the neural network. Further, at block 102, the processor of the monitor determines whether the oxygen saturation correction output is within a first threshold for a maximum point value and a second threshold for a minimum point value. The threshold minimum point value may be, for example, half of a point, and the threshold maximum point value may be, for example, four points. If the processor determines the oxygen saturation correction output is within the first threshold for the maximum point value and the second threshold for the minimum point value, the processor of the monitor proceeds to block 104.

At block 104, the processor of the monitor adjusts the calculated oxygen saturation level based on the oxygen saturation correction output by the neural network to generate a corrected oxygen saturation value. However, if at block 102, the processor determines the oxygen saturation correction output is not within the first threshold for the maximum point value and the second threshold for the minimum point value, the processor of the monitor returns to block 96 and performs the method 90 as described above. In some embodiments, the processor of the monitor may input the calculated oxygen saturation value into the neural network. The neural network may then automatically generate the corrected oxygen saturation value based on the one or more coefficients, the one or more inputs, and the calculated oxygen saturation value. Accordingly, the processor of the monitor may efficiently generate the corrected oxygen saturation value for a particular patient based on the signals generated by the sensor positioned at a measurement site on the patient.

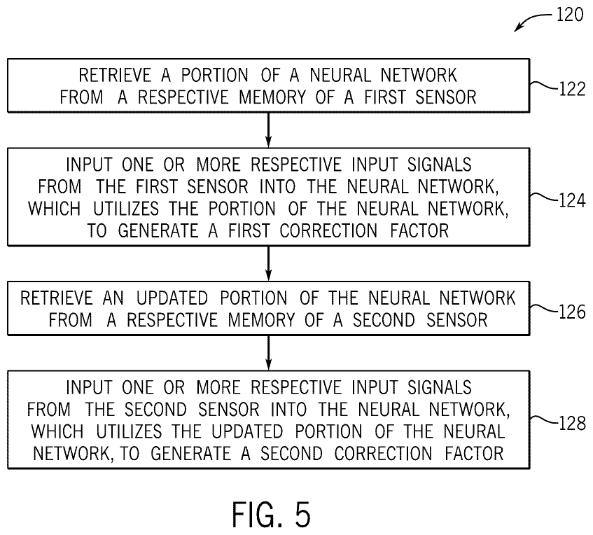

FIG. 5 is a flow diagram of an embodiment of a method 120 for generating a second oxygen saturation correction (e.g., a second correction factor), in accordance with an aspect of the present disclosure. The method 120 disclosed herein includes various steps represented by blocks. It should be noted that at least some steps of the method 120 may be performed as an automated procedure by a system, such as the medical monitoring system 10 of FIG. 1 or a cloud computing system. Further, certain steps or portions of the method 120 may be performed by certain devices, such as the monitor 12 (e.g., the processing system that includes the processor 40) and/or the sensor 14 (e.g., the processing system that includes the processor 32). It should be appreciated that “the processing system” as used herein may refer to components (e.g., the processor 40, the processor 32) in the monitor 12, the sensor 14, or both. Although the flow diagram illustrates the steps in a certain sequence, it should be understood that the steps may be performed in any suitable order and certain steps may be carried out simultaneously, where appropriate.

At block 122, the processor of the monitor retrieves a portion of a neural network from a respective memory of a first sensor. As an example, a sensor identifier, a weight and bias, and/or any other suitable neural network data may be considered to be a part of the portion of the neural network. At block 124, the processor of the monitor inputs one or more respective input signals from the first sensor into the neural network, which utilizes the portion of the neural network, to generate a first correction factor. For example, coefficient selection for the neural network may be based on the sensor identifier and/or the weight and bias may be input into the neural network.

At block 126, the processor of the monitor retrieves an updated portion of the neural network from a respective memory of a second sensor. For example, the second sensor may be associated with a new sensor family, a sensor type, and/or a change in an existing sensor family. As such, the updated portion of the neural network is updated based on the new sensor family, the sensor type, and/or the change in an existing sensor family. Additionally or alternatively, the updated portion of the neural network may be updated based on recognition of improvements to the neural network at a later time. At block 128, the processor of the monitor inputs the one or more respective input signals from the second sensor into the neural network, which utilizes the updated portion of the neural network, to generate the second correction factor. Indeed, the updated portion of the neural network may result in updates to the neural network, which causes the processor of the monitor to generate the second correction factor based on the updates.

As such, embodiments described herein enable adjustment of the oxygen saturation value based on the oxygen saturation correction output by the neural network to generate the corrected oxygen saturation value. In this manner, the monitor may account for errors caused by varying physiological factors of different patients, such as light scattering and absorption. Indeed, it is presently recognized that the physiological factors may cause a path of light through the tissue of a particular patient to change, which may result in pathlength errors. The pathlength errors may impact oxygen saturation measurements or lead to inaccurate readings of oxygen saturation by affecting accuracy of absorption of light. Thus, the systems and methods described herein enable generation of the corrected oxygen saturation value based on the one or more input signals derived from the sensor and the one or more coefficients (e.g., selected based on the sensor information, which may be stored and/or updated on the sensor) to enable the monitor to provide and/or display the corrected oxygen saturation value to a clinician and/or the patient, which may be a more accurate value. Therefore, the clinician and/or the patient may obtain an accurate reading of the oxygen saturation value of the blood of the patient to enable appropriate health monitoring and management.

Advantageously, the systems and methods described herein enable generation of the corrected oxygen saturation value without calculating or estimating the parameters of the skin of the patient (e.g., without calculating or estimating melanin content of the skin of the patient). Instead, the systems and methods described herein enable generation of the corrected oxygen saturation value based on the one or more input signals derived from the sensor, wherein at least one of the one or more input signals indicate an amount of blood at the measurement site on the patient, as well as the one or more coefficients (e.g., selected based on the sensor information, which may be stored and/or updated on the sensor).

Although the neural network is described herein as being employed by the processor of the monitor to output the oxygen saturation correction, it should be noted that any suitable method may be employed to output the oxygen saturation correction. For example, the processor of the monitor may employ a linear regression model to output the oxygen saturation correction. As another example, the processor of the monitor may employ a decision tree to output the oxygen saturation correction. Therefore, any suitable method for deriving the oxygen saturation correction based on the one or more input signals and the one or more coefficients may be employed by the processor of the monitor.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A medical monitoring system, comprising:
a sensor comprising a sensor memory that stores a sensor identifier, a weight and bias, or any combination thereof; and
a monitor comprising:
a port to communicatively couple to the sensor to receive a sensor signal, the sensor identifier, the weight and bias, or any combination thereof;
a display; and
monitor processing circuitry to:
select a coefficient based on the sensor identifier;
input the coefficient, the sensor signal, the weight and bias, or any combination thereof to a neural network to output a correction factor; and
adjust a physiological parameter based on the correction factor.

2. The medical monitoring system of claim 1, wherein the sensor identifier comprises data associated with a type of the sensor.

3. The medical monitoring system of claim 1, wherein the sensor signal comprises a signal output by the sensor based on light detected by a detector of the sensor, and the physiological parameter comprises oxygen saturation.

4. The medical monitoring system of claim 3, wherein the monitoring processing circuitry determines one or more input signals based on the sensor signal, and the one or more input signals comprise a ratio of an alternating current (AC) component, a ratio of a direct current (DC) component, a ratio-of-ratios, red percent modulation, infrared (IR) percent modulation, or any combination thereof.

5. The medical monitoring system of claim 4, wherein the monitor processing circuitry inputs at least one of the one or more input signals to the neural network to output the correction factor.

6. The medical monitoring system of claim 3, wherein the sensor memory stores a power transfer unit (PTU) characterization, and the monitor processing circuitry receives the PTU characterization and inputs the PTU characterization to the neural network to output the correction factor.

7. The medical monitoring system of claim 1, wherein the sensor memory comprises an erasable programmable read-only memory (EPROM) that stores the sensor identifier, the weight and bias, or any combination thereof.

8. The medical monitoring system of claim 1, wherein the correction factor comprises a first threshold for a maximum value and a second threshold for a minimum value.

9. The medical monitoring system of claim 1, comprising the monitor processing circuitry to instruct presentation of the adjusted physiological parameter on the display.

10. A method of operating a medical monitoring system, comprising:
receiving, at a processor and from a memory of a sensor, a sensor identifier, a weight and bias, a sensor signal, or any combination thereof;
selecting, via the processor, a coefficient based on the sensor identifier;
inputting, via the processor, the coefficient, the weight and bias, or any combination thereof to a neural network to output a correction factor; and
determining, via the processor, a physiological parameter based on the sensor signal;
adjusting, via the processor, the physiological parameter based on the correction factor.

11. The method of claim 10, wherein the sensor signal comprises a signal output by the sensor based on light detected by a detector of the sensor, and the physiological parameter comprises oxygen saturation.

12. The method of claim 11, comprising:
determining, via the processor, one or more input signals based on the sensor signal, wherein the input signal comprises a ratio of an alternating current (AC) component, a ratio of a direct current (DC) component, a ratio-of-ratios, red percent modulation, infrared (IR) percent modulation, or any combination thereof; and
inputting, via the processor, at least one of the one or more input signals to the neural network to output the correction factor.

13. The method of claim 10, comprising averaging, via the processor, the correction factor over a period of time.

14. The method of claim 10, comprising adjusting, via the processor, the physiological parameter in response to the correction factor being equal to or above a threshold value.

15. The method of claim 10, comprising instructing, via the processor, display of the adjusted physiological parameter on a display.
